(19) United States
(12) Patent Application Publication
(10) Pub. No.: US 2008/0213341 A1
(43) Pub. Date: Sep. 4, 2008

(54) Butyric Acid Esters of Carbohydrates and Carbohydrate Polyols

(76) Inventors: Alireza Haji Begli, Ramsen (DE); Michael Klingeberg, Grunstadt (DE); Jorg Kowalczyk, Eiscnberg/Steinborn (DE); Gunhild Kozianowski, Grunstadt (DE); Stephan Theis, Bad Durkheim (DE); Wolfgang Wach, Worms (DE)

Correspondence Address: OSTROLENK FABER GERB & SOFFEN, 1180 AVENUE OF THE AMERICAS, NEW YORK, NY, 100368403, US

(21) Appl. No.: 12/088,396

(22) PCT Filed: Sep. 28, 2006
(86) PCT No.: PCT/EP2006/009436

§ 371 Date: Apr. 8, 2008

(30) Foreign Application Priority Data
Sep. 28, 2005 (DE) 10 2005 046 237.5

(51) Int. Cl. A61K 9/00; A61K 31/19; A61K 31/047; A61P 1/00; A61K 9/10

(52) U.S. Cl. 424/439; 514/557; 514/23; 424/490

(57) ABSTRACT

The invention relates to butyric acid esters or butyrate esters of carbohydrates and carbohydrate polyols and their use as a butyrate carrier and butyrate source for the gastrointestinal tract, in particular for the prevention and treatment of diseases of the gastrointestinal tract, especially of the large intestine.

The invention relates to butyric acid esters or butyrate esters of carbohydrates and carbohydrate polyols and their use as a butyrate carrier and a butyrate source for the gastrointestinal tract, in particular for the prevention and treatment of diseases of the gastrointestinal tract, especially of the large intestine.

The C₄ acid butyric acid (butyrate) in the large intestine of a mammal in general originates from the fermentation of undigested food constituents, in particular of undigested carbohydrates, due to the microbial flora of the large intestine. Butyrate is the dominating energy source for the epithelial cells, in particular epithelial cells of the posterior large intestine. In addition to its importance as an energy substrate for the colonocytes, butyrate also influences physiological functions, such as cellular proliferation, differentiation and apoptosis, and plays a central role as a growth factor for a healthy intestine or epithelium and in the maintenance of the mucosal barrier in the large intestine. Butyrate contributes to the detoxification of possible mutagenic metabolic products in the large intestine and acts against oxidative stress, for example via the induction of the gene expression protective proteins such as intestinal glutathione S transferase or the inhibition of ornithine decarboxylase. Furthermore, butyrate has a controlling effect on the induction of specific genes of the cell cycle regulation, antibacterial peptides and signal cascades.

A lack of short-chain fatty acids such as butyrate is associated with various inflammatory, infectious and malignant diseases of the intestine. A lack of butyrate can lead to inflammation of the colon, for example to diversion colitis or pseudomembranous colitis (Rombeau, et al. 1995). In pseudomembranous colitis, which occurs in 1-2% of all taking of antibiotics, a severe syndrome occurs, which is especially caused by infection with the bacterium Clostridium difficile. Butyrate moreover has importance in the prophylaxis of infection in antibiotic-associated diarrhea, which occurs in 10 to 20% of all taking of antibiotics, and in traveler's diarrhea, which occurs in 25% of all travelers to the Mediterranean and 50% of all travelers to the tropics or subtropics. Butyrate also plays a role in the pathogenesis of other inflammatory diseases of the large intestine, such as ulcerative colitis. The decreased luminal availability of butyrate is regarded as a factor for such diseases (Cummings, 1995).

The prevalence of chronic inflammatory intestinal diseases such as ulcerative colitis is estimated at about 0.1%, just as the prevalence of Crohn's disease. The prevalence for irritable bowel syndrome of 1.1% is additionally a multiple higher. The provision of butyrate in the large intestine counts in these diseases as a suitable measure for maintenance of remission and also for the improvement of wound healing, for example after intestinal operations (Wächterhäuser et al., 2000). Butyrate also counts as a significant protective factor for colorectal carcinoma, whose incidence is estimated at about 0.1%, and for the development of carcinoma.

High butyrate concentrations in the large intestine, in particular in posterior regions of the large intestine, in which inflammatory, infectious and malignant intestinal diseases are essentially located, support a healthy intestinal medium and a healthy intestinal epithelium, improve symptoms of ulcerative information of the colon, so for the prophylaxis of infections and are an essential protective factor for colorectal carcinoma, that is they reduce the risk of suffering from cancer of the large intestine. On account of the high incidences, infectious, inflammatory and malignant diseases of the intestine cause considerable health costs. Measures for the assistance of intestinal health by provision of adequately high amounts of butyrate in the large intestine, in particular also in posterior regions of the intestine, therefore have a great economical potential for the reduction of the health costs for these diseases.

In addition to the general provision of butyrate for the large intestine, up to now the availability over the entire length of the large intestine and particularly in specific regions such as the posterior intestinal regions is especially critical (Scheppach et al., 1992). Known means for making butyric acid available to the large intestine are oral administration or the consumption of indigestible carbohydrates. Indigestible carbohydrates of food such as the resistant starch or pectin used in foods could be starting substances for the formation of butyrate after fermentation by the microflora of the large intestine. Cummings et al. (1996), however, report on considerable individual differences in the fermentative formation of butyrate; some people are not able to form butyrate from indigestible carbohydrates by microbial fermentation in the large intestine.

The proportion of butyrate formed in fermentation can vary greatly. It is dependent on the particular fermented carbohydrate. Little butyrate, but mainly acetate, propionate and gases such as hydrogen, carbon dioxide and methane are formed from various indigestible carbohydrates such as inulin, polydextrose, pectin or arabinogalactan (Cummings et al., 2001).

The concentration of butyrate that is formed in the fermentation of indigestible carbohydrates is high in the anterior large intestine, but decreases towards the posterior colon (Cummings et al., 1995). For the assistance of intestinal health and as a protective factor, high butyrate concentrations are advantageous over the entire large intestine, especially also in the posterior regions.

Up to about 5 g/day of butyrate are regarded as a preventively or therapeutically active amount of butyrate (Scheppach et al., 1992). According to Vernia et al. (2000), 4 g/day of butyrate assist the therapeutic effect of mesalazine (2.4 g/day) in the treatment of ulcerative colitis. This is an amount which cannot be achieved by the fermentative degradation of carbohydrates in the large intestine. Thus it is known, for example, that with a degradation of 30 g/day of fermentable substrate (bulk materials, resistant starch and the like), the formation of on average 2.2 g of butyrate occurs (Wolin, 1981). Per 1 g of known fermentable substrates, accordingly only approximately 0.07 g of butyrate is formed.

It would be advantageous to make available larger amounts of butyrate in the large intestines via the food. Free, that is unbound, butyrate, which is absorbed via the food, is rapidly and completely absorbed in the small intestine and thus does not pass into the large intestine (Schmitt et al., 1976). In the food technology field, the possibilities of use of pure free butyric acid are restricted. It is also disadvantageous that free butyric acid is a generally sensorially, gustatorily and organoleptically unattractive substance and cannot be employed directly in foods or drinks. It is known for tributyrin (triglyceride containing 3 molecules of butyrate) that after oral administration it is rapidly cleaved in the anterior small intestine and resulting butyrate is already absorbed in the small intestine (Gaschott et al., 2001).

Orally administered tributyrin does not pass into the small intestine and proves to be inactive in the prevention of colon cancer (Newmark et al., 1994).

Alternatively, the intravenous administration of butyric acid and of butyric acid esters based on xylitol and other monosaccharides and monosaccharide derivatives as (pro)drugs is described (Desmet et al., 1991, Pouillart et al., 1992, Santini et al., 1998). The release of butyrate in the large intestine is not described in these studies. However, intestinal lavages (enemas) with small-chain fatty acids such as butyric acid are known (Scheppach et al., 1992).

The technical problem underlying the present invention thus consists especially in making available means and measures in order, particularly by simple oral or central administration, to administer therapeutically or preventively adequate amounts of butyrate to the large intestine.

The technical problem is solved by the use of a least one of butyric acid ester or of a mixture of a least two different butyric acid esters (butyric acid ester mixture) as a butyrate source in the digestive tract of the human or animal body, especially for the treatment and/or prevention of or against diseases of the gastrointestinal tract, particularly the anterior and/or posterior intestinal sections, in particular of the large intestine, but particularly of the colon. According to the invention, the butyric acid esters are esters of a least one carbohydrate, esters of a least one carbohydrate polyol and/or esters of mixtures of carbohydrate and carbohydrate polyol. The butyric acid ester(s) used according to the invention is/are administered orally or enterally, particularly in micro- and/or macroencapsulated form.

It has surprisingly been found that esters of butyric acid (n-butanoic acid) with carbohydrates and/or with carbohydrate polyols, thus saccharides or saccharide alcohols, can be employed as small intestinal-stable carriers for butyrate. Certain butyric acid esters of carbohydrates and carbohydrate polyols are surprisingly stable on passage through the stomach and small intestine. They are not degraded by enzymes such as lipases and esterases present in the small intestine, and no butyrate is released from them there. The butyrate bound in this way thus escapes digestion and absorption in the small intestine. Advantageously, the butyric acid esters used according to the invention pass into the large intestine unchanged. The esterified butyric acid is then released from the butyric acid esters there by the microbial activity of bacteria established in the large intestine.

It is seen here especially that
a) with an increasing degree of substitution (DS) of the butyric acid esters the metabolization and release of butyric acid becomes lower; completely esterified derivatives surprisingly cause a lower release of the butyric acid in the large intestine than partially esterified derivatives;
b) the butyric acid release in the large intestine with pure butyric acid derivatives is surprisingly higher than in the mixed acid derivatives of acetate and butyrate;
c) the release of butyric acid in the large intestine surprisingly depends on the nature of the carbohydrate or carbohydrate polyol used and also on the level of the degree of esterification;
d) the butyric acid esters of the C₅-polyols such as xylitol, the C₆-polyols such as sorbitol and the disaccharide polyols such as isomalt, in each case with a DS of 3 to 4, surprisingly lead to a particularly high release of butyric acid in the large intestine.

As also explained in the examples below, only approximately 0.3 g of butyrate results, for example, from the fermentation of sorbitol per 1 g of substrate, and in each case approximately 0.2 g and approximately 0.3 g respectively of butyrate from the fermentation of the unesterified carbohydrate polyols isomalt and xylitol. In the degradation of other fermentable carbohydrates such as resistant starch, approximately 0.2 g of butyrate results per 1 g of substrate. As opposed to that, in the case of tributyrylsorbitol and tetrabutyrylsorbitol which are preferred according to the invention surprisingly about 0.7 g of butyrate is released per 1 g of substrate. Likewise, up to 0.7 g of butyrate per 1 g of substrate is likewise also formed from tributyrylxylitol and tetrabutyrylxylitol which are preferred according to the invention. With butyric acid esters according to the invention, around 3 times more butyrate is surprisingly and advantageously formed in comparison to nonesterified substrates. If they are employed according to the invention, the butyric acid esters “yield” significantly more butyrate in the large intestine a) on account of the release of the esterified butyrate radicals and preferably b) on account of the fermentative degradation of the carbohydrate and carbohydrate polyol radical respectively than can be formed by the purely fermentative degradation of known nonesterified substrates.

The butyric acid esters of carbohydrate polyols used according to the invention are particularly suitable for administering the therapeutically and preventively necessary amounts of butyrate of approximately 0.5 to 5 g/day to the large intestine. These amounts of butyrate could already be achieved with an uptake of 0.7 to 7 g/day of butyric acid esters such as tri- and tetrabutyrylsorbitol, and tri- and tetrabutyrylxylitol. For the same amount of butyrate, unesterified substrates must be administered in amounts of up to 70 g, that is nearly 10 times more substance, by means of fermentative degradation of carbohydrates such as resistant starch.

The results of the incubation experiments with colon bacteria of humans show that the butyric acid esters used according to the invention are surprisingly only metabolized slowly. That is to say that butyrate is released and formed over a long period, particularly over more than 72 h. The butyric acid esters used according to the invention are thus a continual and long-lasting source for butyrate in the large intestine. Owing to the slow release, it is guaranteed that butyric acid esters used according to the invention are present during the entire large intestinal passage and thus also pass into posterior large intestinal regions. Adequately large amounts of butyrate are thereby advantageously also available in posterior large intestinal regions and can thereby display prophylactic and therapeutic effects respectively in those intestinal regions where the inflammatory, infectious or malignant diseases occur most.

Preferably, the butyric acid ester used according to the invention has a degree of substitution (DS) of 3 to 4. DS of 1, 2, 3, 4, 5, 6, 7, 8 and 9 are likewise preferred, depending on application area and starting compound. Preferably, the butyric acid ester is partially esterified and preferably has a degree of esterification of 10 to 90%, 30 to 90%, 40 to 80% and in particular of 50 to 80%. Degrees of esterification of at least 10, 20, 30, 40, 45, 50, 55, 60, 65, 70, 75, 80, 85, 90 or 95% and/or at most 10, 20, 30, 40, 45, 50, 55, 60, 65, 70, 75, 80, 85, 90 or 95% are likewise preferred, depending on application area and starting compound.

In a preferred variant, the carbohydrate or the carbohydrate polyol is a monosaccharide, disaccharide, oligosaccharide or polysaccharide. Mixtures of a least one carbohydrate and at least one carbohydrate polyol, mannitol, sorbitol, xylitol, lactitol, maltitol, erythritol, isomalt, 1,6-GPS, 1,1-GPS, 1,1-GPM, hydrogenated starch hydrolyzate, hydrogenated glucose syrup and/or a mixture thereof are also preferred. The carbohydrate polyol is preferably a C₅-polyol and/or a C₆-polyol. In a further preferred variant, the carbohydrate polyol is a disaccharide polyol.

In a preferred embodiment, the butyric acid ester used according to the invention is tributyrylsorbitol, tetra-butyrylsorbitol, tributyrylxylitol, tetrabutyrylxylitol, pentabutyrylisomalt or a mixture thereof and/or a mixture with other butyric acid esters. Butyric acid isomalt esters are also distinguished by their only slightly unpleasant taste, in comparison to other butyric acid esters used according to the invention, for example butyric acid sorbitol ester.

The invention also relates to the use of mixed esters with butyrate and other short-chain alkanoic acids, preferably acetate.

The alkanoic acid esters, that is butyric acid esters and others, are prepared in a manner known per se, preferably by reaction with alkanoic acid or acid anhydride.

The molar ratio of carbohydrate and/or carbohydrate polyol to acid or acid anhydride, according to the DS to be achieved, is as a rule from 1:1 to 1:10, especially from 1:3 to 1:6. The esterification can be carried out in the presence of an acidic catalyst such as tin oxalate. Excess acid is subsequently removed in a manner known per se.

A preferred subject of the invention is the use of the butyric acid ester characterized above for the prophylactic and/or therapeutic support of a healthy intestinal medium and of a healthy intestinal epithelium, and for the treatment or prevention of diseases of the gastrointestinal tract of the human or animal body.

Diseases of this type are illnesses which are treated or can be prevented by an administration of butyrate to the large intestine. These especially include illnesses which accompany a butyrate deficiency in the gastrointestinal tract, illnesses and conditions in which butyrate contributes to the detoxification of possible mutagenic metabolic products in the large intestine, illnesses and conditions in which butyrate counteracts oxidative stress, for example by means of the induction of the gene expression of protective proteins such as intestinal glutathione S transferase or the inhibition of ornithine decarboxylase, illnesses which are accompanied by a pathogenic, toxic or medicament-related impairment or damage to the epithelial cells and colonocytes of the posterior large intestine or to the mucosal barrier in the large intestine, inflammations of the colon such as diversion colitis or pseudomembranous colitis, chronic inflammatory intestinal diseases such as ulcerative colitis and Crohn's disease and irritable bowel syndrome, infections with clostridia, antibiotic-associated diarrhea, traveler's diarrhea and colorectal carcinoma. The use according to the invention also provides for remission maintenance and the improvement of wound healing, for example after intestinal operations.

Accordingly, the invention provides for the use of at least one of the butyric acid esters or of a butyric acid ester mixture as an active substance, in particular as a therapeutic active substance. These are preferably employed in medicaments, pharmaceutical compositions or pharmaceutical-like preparations. When used as an active substance, the butyric acid esters are preferably employed together with other pharmacologically suitable vehicles, additives and excipients, such as lubricants, mold-release agents, thickening agents, emulsifiers, stabilizers, preservatives, lecithin, intensive sweeteners, sweetening agents, colorants, flavorings, aromatic substances, coating materials and/or fillers.

According to the invention, use preferably takes place in solid and/or liquid, in particular in suspended or dissolved, form. Suitable suspending agents and solvents are preferably food-compatible solvents and emulsifiers, including water, alcohols and mixtures thereof. Administration preferably takes place—depending on application area—in the form of a suspension, solution, emulsion, drops, juices, tablets, pills, capsules, pastilles, coated tablets, jellies, granules, powder, injection or infusion solution or in combinations thereof. Use can also take place in a further, similarly suitable presentation form.

According to the invention, the administration of butyric acid esters in microencapsulated or macroencapsulated form to the human or animal body is particularly preferred.

Butyric acid esters can thereby be administered in organoleptically neutral form.

Taste-neutral transport is especially made possible by the digestive tract. Preferably, at least one butyric acid ester is present in micro- and/or macroencapsulated form. Encapsulation makes possible an improved, especially chronologically delayed or steady and continuous release of the butyric acid esters at the target site in the gastrointestinal tract or large intestine. The application area can be further extended by encapsulation; it is possible to prepare dry mixtures and easily dosable systems which contain the butyric acid esters or mixtures according to the invention. Sensorially negative effects of the butyric acid esters according to the invention, which are present in some cases, can be largely concealed by encapsulation.

Presently, “encapsulation” of the butyric acid esters is understood as meaning encapsulation which is especially based on the binding of the butyric acid esters to a carrier, for example by means of adsorption, covalent or ionic bonding or linkage with bi- or multifunctional reagents. Encapsulation is further preferably also based on the inclusion of the butyric acid esters in a matrix or membrane such as, for example, by ionotropic gel formation, polyelectrolyte complexes, simplexes, cold gelation, formation of hydrocolloids, polymerization and/or solvent precipitation.

Preferably, at least one butyric acid ester is encapsulated in at least one shell material by inclusion processes.

As materials for the encapsulation of the butyric acid esters according to the invention, at least one agent selected from alginates, agar-agar, agarose, pectins and pectinates, guar gum, chitosan, cellulose derivatives, starch derivatives, gum arabic, waxes, mono-, di- and triglycerides and other organic and inorganic substances, is preferably employed. In a particularly preferred embodiment, polysaccharides, in particular pectinate and alginate, are employed for encapsulation. The forms of the micro- and macroencapsulation of the butyric acid esters are preferably selected from spheres, cylinders, fibers or films, tablets, granules, powders, pills, pastilles, coated tablets and jellies.

Preferably, after charging of the butyric acid ester at least one further protective layer of pure carrier material is also put on in order to complete the microencapsulation outwardly. Alternatively, coating is carried out in a known manner with a customary coating material used in pharmacy, for example hydroxypropylmethylcellulose (HPMC). Particularly preferably, a fluidized bed agglomerator is employed for fluidized bed drying.

Microencapsulated butyric acid esters are preferably produced by means of atomizers, for example by means of the blowoff, electrostatic or vibration process, by means of rotating disks and or/or nozzles and jet cutters. Particularly preferably, polysaccharides, preferably pectinates and alginates, together with a butyric acid ester or butyric acid ester mixture in a solution are used for this and the solution obtained is instilled into a precipitation solution by means of atomizers, preferably by means of jet cutters. The precipitation solution employed is preferably calcium chloride solution or magnesium chloride solution. The resulting beads (spheres) have a diameter of less than 50 μm. Preferably, the beads are subsequently dried in a fluidized bed dryer.

Suitable production processes for microencapsulation are furthermore essentially the processes known in the food industry such as spray drying, freeze drying, fluidized bed drying, fluidized bed agglomeration or extrusion.

In spray drying, a syrup of a wall material, for example sugar, polyols and also maltodextrin or other well-crystallizing starch products, in combination with a butyric acid ester or butyric acid ester mixture, is dried by spraying the syrup in a preferably continuous process such that a solid, pulverulent or granule-like product is obtained, in which the butyric acid ester is present encapsulated in the selected wall material.

In the freeze-drying, wall material, for example a sugar, polyol or a maltodextrin, is brought together into solution or suspension with a butyric acid ester or butyric acid ester mixture. After shock-freezing and removal of the water in a manner known per se, a powder is obtained in which the butyric acid esters are embedded.

In microencapsulation by means of fluidized bed drying, wall material is introduced as a finely ground powder, for example sugar, polyol or maltodextrin, and subsequently sprayed with an aqueous solution or suspension of the butyric acid ester or butyric acid ester mixture. In a preferred embodiment, the nozzles for spraying the solution or suspension are above the fluidized bed (topspray method). In an alternative embodiment, the nozzles are integrated in the base of the fluidized bed unit (bottomspray method). By means of the fluidized bed drying, particles according to the invention are built up which contain up to 20% of the butyric acid ester or butyric acid ester mixture.

Preferably, at least one of the butyric acid esters characterized above is employed as the sole butyrate source, as the sole therapeutic or prophylactic active substance. In a further preferred variant, butyric acid ester is employed for the treatment or prevention of these diseases together with at least one further butyrate source and/or at least one further active substance.

Use preferably takes place according to a treatment plan, a therapeutically and/or prophylactically efficacious dose being administered repeatedly as a single dose or multiple doses, for example divided over the course of the day and preferably over a certain period. Preferably, a dose of 0.7 to 7 g/day is administered 2 to 5 times per day in a single dose or in subdoses. On account of the slow release of butyrate found, the appropriately multiplied daily dose can also be administered every 2, 3 or 4 days, preferably every 72 hours. In the case of the dose, an adult human of 75 kg bodyweight is especially assumed, for children and for use in animals the doses must be adjusted appropriately.

In a preferred variant, use in combination with a least one further active substance, in particular in the sense of a combination therapy, for example for the treatment of diseases such as manifest colon carcinoma, by means of chemotherapy (for example with 5-fluorouracil) is provided.

The invention also relates to a procedure for the treatment or prevention of the diseases, in particular of the gastrointestinal tract, comprising the preferably oral or enteral administration of the butyric acid ester to the human or animal body, preferably in a therapeutically or prophylactically efficacious dose.

The invention also relates to a pharmaceutical composition comprising at least one of the butyric acid esters characterized above as at least one active substance as a medicament in the treatment or prevention of the diseases, in particular of the gastrointestinal tract, of the human or animal body.

The invention also relates to the use of the butyric acid ester characterized above as an active substance in foodstuffs, foods, luxury foods and animal feeds and preferably for the production of a medicament for the treatment or prevention of the diseases, in particular those of the gastrointestinal tract, of the human or animal body.

In connection with the invention, “foodstuffs” are understood especially as meaning all foods and luxury foods and animal feeds, which in particular have a nutritional value and which can be used for the partial or complete nutrition of the human or animal body. Among these are also understood special foods such as baby food, dietetic foods, stomach tube food for central nutrition and the like. Among these are also understood animal feeds, that is all types of food for animals both in the small animal and in the large cattle area, such as agricultural productive animals, sport horses, but also domestic, zoo and luxury animals. According to this invention, the foodstuff is present as a concentrate, as a base material or as a semifinished product. Foodstuffs are presently also understood as meaning drinks such as alcohol-free drinks, soft drinks, effervescent soft drinks, fruit juice drinks, lemonade, energy drinks, fruit juices, grape juice, fruit nectar, coffee, cocoa, milk, mineral drinks, tea and infusion drinks and alcoholic drinks such as beer, nutrient beer, beer mixed drinks, sour milk drinks (kefir, kumys and others), wine, fruit wine (apple wine and others).

The invention also relates to a composition, particularly a foodstuff, feed or medicament, comprising at least one butyric acid ester or a butyric acid ester mixture used according to the invention and characterized above. In preferred embodiments, at least one further constituent is present in the composition; this is selected from:
carbohydrate polyols, preferably mannitol, sorbitol, xylitol, lactitol, maltitol, erythritol, isomalt, 1,6-GPS, 1,1-GPS, 1,1-GPM, hydrogenated starch hydrolyzates, hydrogenated glucose syrups and mixtures thereof;
carbohydrates, preferably monosaccharides, di-saccharides, oligosaccharides, polysaccharides and mixtures thereof;
soluble and/or insoluble bulk materials, in particular resistant starch, modified starch, polydextrose, fructo-oligosaccharides, galactooligosaccharides, trans-galactosylated oligosaccharides such as 6′-galactosyl lactose or 4′-galactosyllactose, lactulose, lactobionic acid, maltobionic acid, xylooligosaccharides, lacto-sucrose, maltooligosaccharides, isomaltooligo-saccharides, gentiooligosaccharides, glucosylsucrose, soybean oligosaccharides, chitooligosaccharides, chitosan oligosaccharides, pectin, pectin oligo-saccharides, condensed oligosaccharides, caramel products, galactomannan oligosaccharides, fucose-containing oligosaccharides, fucose derivative-containing oligosaccharides, pyrodextrin, partially hydrolyzed guar gum, a variant thereof obtained by partial hydrolysis, hydrogenation, oxidation, enzymatic or chemical modification of saccharides, and fibrous substances, in particular from oats, wheat, vegetables such as the tomato or pea, fruit such as apples and fruit berries, sugar beet, fruit of the carob tree or cellulose;
short-chain fatty acids, preferably butyric acid, propionic acid, acetic acid, lactic acid;
butyric acid glycerol esters, preferably glycerol tri-butyrate, glycerol dibutyrate, glycerol monobutyrate; and
acylated starch, preferably butyrylated starch.

In a further variant, the composition contains a mixture of at least two of the aforementioned components, in particular of a least one carbohydrate and at least one carbohydrate polyol.

The invention also relates to a foodstuff of this type, selected from:
dairy produce and dairy products such as cheese, butter, yoghurt, kefir, quark, sour milk, buttermilk, cream, condensed milk, dried milk, whey, milk sugar, milk protein, mixed milk, half-fat milk, mixed whey or butterfat products or preparations;
butterfat products, mixed fat products, edible fats and edible oils;
blancmange, cream, mousse and other desserts;
baked products such as bread including cookies and fine baked products, long-life baked products, cookie products and waffles;
spreads, in particular fat-containing spreads, margarine products and shortening;
instant products and broth products;
fruit products or preparations such as jams, marmalades, jellies, fruit preserves, fruit pulp, fruit pith, fruit juices, fruit juice concentrates, fruit nectar and fruit powder;
cereals, muesli and cereal mixtures, and ready prepared cereal-containing products such as muesli bars and breakfast products;
nonalcoholic drinks, drink basic materials and drink powder; and
confectionery such as chocolates, hard caramels, soft caramels, chewing gum, candy, fondant products, jelly products, licorice, marshmallow products, flakes, compressed sweets, candied fruit, brittle, nougat products, ice cups, marzipan, ice cream.

The invention also relates to a dietetic special food and enteral food derived therefrom.

The invention finally also relates to a composition comprising at least one of the butyric acid esters characterized above as a feed such as animal food, premixes for animal food, starch-rich feed, concentrated feed and mash. Especially in animal breeding and the meat industry, prophylaxis and therapy of intestinal diseases can thus be harmlessly made possible or assisted for humans and animals.

The invention preferably provides for the use of the butyric acid esters or mixtures thereof in products for enteral nutrition and as a food supplement or dietary supplement.

The invention is illustrated in more detail by means of the following examples and figures; these are not to be understood as being restrictive.

The figures show:

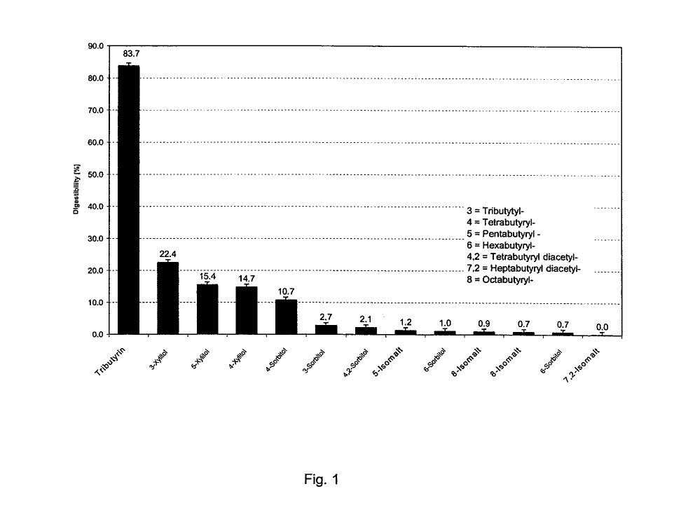

FIG. 1 Chart of the digestibility of various butyric acid xylitol, sorbitol and isomalt esters.

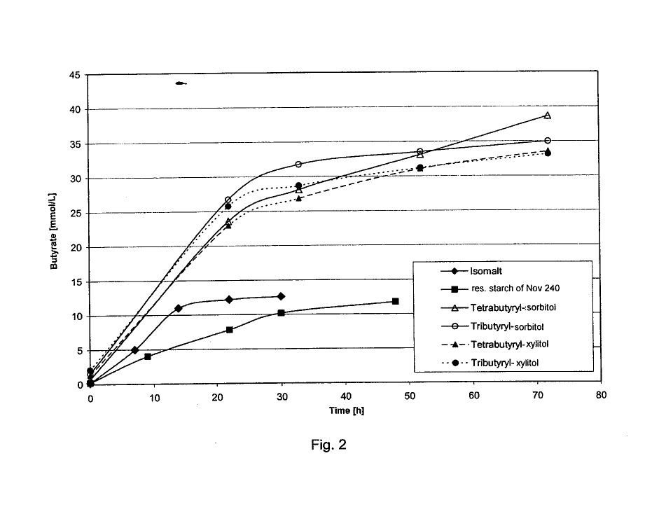

FIG. 2 Graph of butyrate formation during in vitro fermentation with intestinal bacteria as a function of time when using different substrates.

### EXAMPLE 1

### Butyric Acid Ester Based on Sorbitol (DS 3)

100 g of sorbitol (0.55 mol) were suspended in 260.6 g of butyric anhydride (1.65 mol) in a stirring vessel. After heating the suspension to 160° C., the mixture was stirred for a further 2 hours. After reaction was complete, the excess acid was removed in vacuo. A clear pale yellow syrup was obtained as the product.

### EXAMPLE 2

### Butyric Acid Ester Based on Sorbitol (DS 4)

100 g of sorbitol (0.55 mol) were suspended in 347.4 g of butyric anhydride (2.2 mol) in a stirring vessel. After heating the suspension to 160° C., the mixture was stirred for a further 2 hours. After reaction was complete, the excess acid was removed in vacuo. A clear pale yellow syrup was obtained as the product.

### EXAMPLE 3

### Butyric Acid Ester Based on Sorbitol (DS 6)

100 g of sorbitol (0.55 mol) were suspended in 521.1 g of butyric anhydride (3.3 mol) in a stirring vessel. After heating the suspension to 160° C., the mixture was stirred for a further 4 hours. After reaction was complete, the excess acid was removed in vacuo. A clear pale yellow syrup was obtained as the product.

### EXAMPLE 4

### Butyric Acid Ester Based on Sorbitol (DS 4)

100 g of sorbitol (0.55 mol) were suspended in 290.2 g of butyric acid (3.3 mol) in a stirring vessel and the mixture was heated to 160° C. The mixture was treated with 0.6 g of tin oxalate at this temperature and stirred under reflux for a further 6 hours. After reaction was complete and removal of the catalyst, the excess acid was removed in vacuo. A clear pale yellow syrup was obtained as the product.

### EXAMPLE 5

### Butyric Acid Ester Based on Sorbitol (DS 6)

100 g of sorbitol (0.55 mol) were suspended in 290.2 g of butyric acid (3.3 mol) in a stirring vessel and the mixture was heated to 160° C. The mixture was treated with 0.6 g of tin oxalate at this temperature and stirred under reflux for a further 6 hours. After this, the batch was treated with 112.1 g of acetic anhydride (1.1 mol) and kept under reflux for a further hour. After reaction was complete and removal of the catalyst, the excess acid was removed in vacuo. A clear pale yellow syrup was obtained as the product.

### EXAMPLE 6

### Butyric Acid Ester Based on Isomalt (DS 5)

50 g of isomalt (0.14 mol) were suspended in 131 g of butyric anhydride (0.83 mol) in a stirring vessel. After heating the suspension to 160° C., the mixture was stirred for a further 2 hours. After reaction was complete, the excess acid was removed in vacuo. A clear pale yellow syrup was obtained as the product.

### EXAMPLE 7

### Butyric Acid Ester Based on Xylitol (DS 3)

100 g of xylitol (0.66 mol) were suspended in 312.2 g of butyric anhydride (1.97 mol) in a stirring vessel. After a reaction time of 2 hours at 160° C., the excess acid was removed in vacuo and the product was isolated as an almost colorless syrup.

### EXAMPLE 8

### In Vitro Digestibility of Butyric Acid Esters of Carbohydrates and Carbohydrate Polyols

For the investigation of the small intestine stability of the butyric acid esters of carbohydrate polyols, the butyric acid esters listed below were incubated with lipases and esterases from the pancreas and the small intestine:
a. Tributyrylxylitol
b. Tetrabutyrylxylitol
c. Pentabutyrylxylitol
d. Tributyrylsorbitol
e. Tetrabutyrylsorbitol
f. Tetrabutyryldiacetylsorbitol
g. Hexabutyrylsorbitol
h. Pentabutyrylisomalt
i. Heptabutyryldiacetylisomalt
j. Octabutyrylisomalt
   Enzyme Preparation from the Small Intestinal Mucosa

Esterases of the small intestine were isolated from pig's small intestine. For this, an 18 m long small intestine of a freshly slaughtered pig was subdivided into 6×3 m sections, and the individual sections were dissected and homogenized in an ultraturrax. After subsequent centrifugation, the esterase was obtained in the soluble supernatant. Esterase activity was detectable over the entire small intestine, the highest activity being located in section 4 (9-12 m).

### Investigation of the Small Intestine Stability

20 mg of tributyrin (control substance) or butyric acid ester were emulsified in 1680 μl of 100 mM phosphate buffer, pH 7.5, together with 4 mg of taurocholic acid, treated with 220 μl of a 0.06% strength pancreatin solution and 100 μl of mucosa supernatant with esterase activity (see above) and incubated at 37° C. for 3 h with stirring. At the end of the reaction, the butyrate released was determined by means of GC.

### Results

The control tributyrin was maximally cleaved under the incubation conditions, i.e. down to the stage of the monobutyrate.

The hydrolysis rates of the individual butyric acid esters including the standard deviation (n=2) are shown in FIG. 1. The completely esterified polyols (hexabutyrylsorbitol, tetrabutyryidiacetylsorbitol, octabutyrylisomalt and heptabutyryldiacetylisomalt) were only hydrolyzed to 0-2.1%. In addition, pentabutyrylisomalt and tributyrylsorbitol also proved resistant to enzymatic degradation. 10.7-22.4% of butyrate were released from tetrabutyrylsorbitol and tri-, tetra- and pentabutyrylxylitol. Altogether, the results show that butyric acid esters of carbohydrate polyols were not or were only insignificantly hydrolyzed during small intestinal passage and were therefore small intestine-stable.

The acid stability in the course of the gastric passage was determined by incubation at 37° C. for 3.5 h at pH 2.0.

Only 0 to 0.43% of the maximally available butyrate were released from a 1% strength suspension to which taurocholic acid was added as an emulsifier. Butyric acid esters are therefore also stable in the stomach.

### EXAMPLE 9

### In Vitro Fermentation of Butyric Acid Esters of Xylitol, Sorbitol and Isomalt Respectively

It was investigated in in vitro fermentation experiments with intestinal bacteria of the human large intestine whether butyric acid esters listed above are hydrolyzed and metabolized by bacteria of the large intestine or their enzymes and butyrate is released in the small intestine.

### Medium

The following medium was used for in vitro fermentation experiments:

| | | |
|---|---|---|
| Tryptone | 1.5 | g/l |
| Yeast extract | 1.0 | g/l |
| KH₂PO₄ | 0.24 | g/l |
| Na₂HPO₄ | 0.24 | g/l |
| (NH₄)₂PO₄ | 1.24 | g/l |
| NaCl | 0.48 | g/l |
| MgSO₄ × 7 H₂O | 0.10 | g/l |
| CaCl2 × 2 H₂O | 0.06 | g/l |
| FeSO₄ × 7 H₂O | 2 | mg/l |
| Resazurin | 1 | mg/l |
| Cysteine/HCl | 0.5 | g/l |
| Vitamin solution (according to DSM 141) | 0.5 | ml/l |
| Trace element solution (according to DSM 141) | 9.0 | ml/l |
| NaHCO₃ | 2.0 | g/l |

pH 7.0

Culturing of Intestinal Bacteria with Butyric Acid Esters

9 ml of the aerobic medium described above were treated with 0.5% (w/v) of the butyric acid ester to be tested and 0.2% (w/v) of taurocholic acid. This medium was subsequently inoculated with 1 ml of a 10% strength feces suspension (mixed feces of three subjects) in anaerobic 50 mmol/l phosphate buffer, pH 7.0, to which 0.5 g/l of cysteine/HCl had been added as a reductant. Hungate tubes were incubated at 37° C. with shaking for 72 h, samples were taken at different times and these were investigated for short-chain fatty acids, lactic acid and pH. The extent of the metabolization of the test substances was carried out by means of the determination of the release of butyrate.

### Results

TABLE 1
Balancing of the butyrate formation in the in vitro fermentation experiment

| | | g of | |
|---|---|---|---|
| | Degree of | butyrate/g | x-fold |
| Substance | esterification | of substrate | increase* |
| Isomalt | — | 0.2 | — |
| Pentabutyrylisomalt | 55% | 0.2 | 1.3 |
| Octabutyrylisomalt | 88% | 0.1 | 0.5 |
| Heptabutyryldiacetylisomalt | 100% | 0.1 | 0.4 |
| Sorbitol | — | 0.3 | — |
| Hexabutyrylsorbitol | 100% | 0.2 | 0.8 |
| Tetrabutyrylsorbitol | 67% | 0.7 | 2.7 |
| Tetrabutyryldiacetylsorbitol | 100% | 0.3 | 1.0 |
| Tributyrylsorbitol | 50% | 0.7 | 2.8 |
| Xylitol | — | 0.3 | — |
| Pentabutyrylxylitol | 100% | 0.4 | 1.5 |
| Tetrabutyrylxylitol | 80% | 0.7 | 2.5 |
| Tributyrylxylitol | 60% | 0.6 | 2.2 |
| Res. Starch from Noverlose 240 | — | 0.2 | — |

*= Increase in the butyrate concentration in the fermentation medium in comparison to the nonesterified carbohydrate or carbohydrate polyol

### a) Fermentation of the Butyric Acid Esters of Isomalt

It is clear from Table 1 that in the fermentation of isomalt approximately 0.2 g of butyrate/g of substrate were formed. In the fermentation of octabutyryl- and heptabutyryldiacetylisomalt, in each case at about 0.1 g of butyrate/g of substrate was obtained, i.e. almost no additional butyrate was released from these substances. In the fermentation of pentabutyrylisomalt, 0.2 g of butyrate/g of substrate was formed.

### b) Fermentation of the Butyric Acid Esters of Sorbitol

In the fermentation of the substances tri- and tetrabutyrylsorbitol, it was already possible to observe a strong increase of the butyrate in the fermentation broths even after a short fermentation time. After 72 hours, values of 0.7 g of butyrate/g of substrate were detected, whereas on fermentation of the nonesterified sorbitol only 0.3 g of butyrate/g of substrate resulted (Table 1). The butyrate formation from tri- and tetrabutyrylsorbitol was thus by more than twofold above that of the fermentation of the unesterified sorbitol.

Hexabutyrylsorbitol was metabolized less by the intestinal bacteria than tri- and tetrabutyrylsorbitol, which is confirmed by the low butyrate formation of 0.2 g of butyrate/g of substrate.

### c) Fermentation of the Butyric Acid Esters of Xylitol

The fermentation of tetrabutyrylxylitol with intestinal bacteria resulted in a butyrate formation of 0.7 g of butyrate/g of substrate compared to 0.3 g on fermentation of the nonesterified xylitol. This value corresponds to a more than two-fold increase in the butyrate formation. Large amounts of butyrate were also released from tributyrylxylitol and the completely esterified pentabutyrylxylitol compared to the nonesterified carbohydrate polyol (Table 1).

The results show that on the use of butyric acid esters of carbohydrate polyols in in vitro fermentation experiments with human intestinal bacteria the butyrate formation can be increased more than 3-fold compared to the butyrate formation which can be achieved by fermentation of nonesterified carbohydrates or resistant starch. Butyric acid esters of carbohydrates and carbohydrate polyols are suitable as small intestine-stable butyrate carriers for the large intestine, tributyrylsorbitol, tetrabutyrylsorbitol and tetrabutyrylxylitol being particularly suitable on account of their high butyrate formation.

### EXAMPLE 10

### Sodium Alginate Encapsulation of Tributyrylsorbitol

3 g of sodium alginate were dissolved in 82 g of completely demineralized water in a stirring vessel with intensive stirring. 15 g of tributyrylsorbitol was added to the solution and homogeneously mixed. The sodium alginate/butyric acid ester mixture was instilled into a 100 mmol/l CaCl₂ solution by means of jet cutters. Beads having a diameter of less than 50 μm resulted. The beads were left in the solution for 2 hours for recrosslinkage, subsequently screened and washed with water. The beads can be used moist or after drying in a fluidized bed dryer.

### EXAMPLE 11

### Pectinate Encapsulation of Tributyrylsorbitol

75 g of a 3% strength (w/w) sodium pectinate solution were heated to boiling for complete dissolution. The solution was subsequently cooled to 40° C. 15 g of tributyrylsorbitol were added to the cooled solution and homogeneously mixed. This mixture was finely sprayed into a 2% strength magnesium chloride hexahydrate solution by means of a spray apparatus. Spheres resulted. These spheres were left in the MgCl₂ solution for 1 hour. The spheres were subsequently screened and washed in water.

### EXAMPLE 12

### Sodium Alginate Encapsulation of Tetrabutyrylsorbitol

3 g of sodium alginate were dissolved in 82 g of completely demineralized water in a stirring vessel with intensive stirring. 15 g of tetrabutyrylsorbitol were added to the solution and homogeneously mixed. The sodium alginate/butyric acid ester mixture was instilled into a 100 mmol/l CaCl₂ solution by means of jet cutters. Beads having a diameter of less than 50 μm resulted. The beads were left in the solution for 2 hours for recrosslinkage, subsequently screened and washed with water. The beads can be used moist or after drying in a fluidized bed dryer.

### EXAMPLE 13

### Pectinate Encapsulation of Tetrabutyrylsorbitol

75 g of a 3% strength (w/w) sodium pectinate solution were heated to boiling for complete dissolution. The solution was subsequently cooled to 40° C. 15 g of tetrabutyrylsorbitol were added to the cooled solution and homogeneously mixed. This mixture was finely sprayed into a 2% strength magnesium chloride hexahydrate solution by means of a spray apparatus. The resulting spheres were left in the MgCl₂ solution for 1 hour. The spheres were subsequently screened and washed in water.

### EXAMPLE 14

### Fluidized Bed Agglomeration

5 kg of finely ground isomalt ST are introduced into a fluidized bed agglomerator as wall material (particle size 90%<50 μm) and fluidized. Subsequently, 1200 g of a spray solution, consisting of a mixture of di-, tri- and tetrabutyrylsorbitol (800 g of ester and 400 g of water) are applied over a period of 1 hour. After this, the spray solution is changed and 500 g of a 20% strength isomalt solution is applied by spraying as the outer layer structure. The product is finally dried to a water content of 5.4%.

### EXAMPLE 15

### Spreads Containing Butyric Acid Esters

### Recipe for Margarine

Allow 95 g of margarine to stand at room temperature in a mixing bowl for 2 hours. Add 5 g of butyrate ester or microencapsulated butyrate ester and mix with a mixing rod for 2 min until a homogenous mass results. The mass is subsequently stored at 6-10° C.

Recipe for hazelnut spread

| | | |
|---|---|---|
| 45 | g | of sugar |
| 10 | g | of hazelnut paste |
| 7.5 | g | of skimmed milk powder |
| 7.5 | g | of cocoa powder |
| 24 | g | of fat |
| 5 | g | of butyrate ester or microencapsulated butyrate ester |
| 1 | g | of lecithin |

Mix the ingredients except for lecithin and butyrate ester. Mill the mass in a 3-roll rolling mill until a fineness of 20 to 25 μm is achieved. Add lecithin and the butyrate ester to the mass. Mix until a homogeneous spreadable mass results.

### EXAMPLE 16

### Blancmange and Creams

Recipe for biscuit cream

| | | |
|---|---|---|
| 50 | g | of sugar |
| 25 | g | of hardened vegetable fat |
| 5 | g | of butyrate ester or microencapsulated butyrate ester |
| 0.47 | g | of salt |
| 0.03 | g | of vanillin |
| 3 | g | of milk powder |
| 6 | g | of cocoa |
| 10.1 | g | of maltodextrin (DE 10) |

Mix the facts and butyrate ester until a smooth paste is present. Add the powdered additives to this and mix for about 10-12 min until a homogeneous mass results.

Recipe for dessert cream

| | | |
|---|---|---|
| 310 | g | of sugar |
| 110 | g | of skimmed milk powder |
| 37 | g | of cornstarch |
| 25 | g | of butyrate ester or microencapsulated butyrate ester |
| 13 | g | of carrageenan |
| 5 | g | of vanilla essence |
| 2500 | ml | of whole milk |

Mix all components except for butyrate ester well with one another. Smoothly stir the powder into one part of the whole milk and add the butyrate ester. Boil the remainder of the milk. Stir the powder mixture into the boiling milk and bring to the boil. Bottle and keep cool until consumption.

### EXAMPLE 17

### Dairy Produce and Dairy Products

Recipe for yoghurt cream with raspberries

| | |
|---|---|
| 450 g | of whole milk yoghurt |
| 300 g | of raspberries |
| 10 g | of gelatin |
| 50 g | of butyrate ester or microencapsulated butyrate ester |
| 50 g | of sugar |
| 20 g | of lemon juice |
| 20 g | of whole milk |
| 100 g | of cream |

Steep the gelatin. Smoothly stir the yoghurt, sugar, butyrate ester, lemon juice and whole milk. Dissolve the gelatine and add. Whip the cream until stiff and draw under the mass. Fill the raspberries into a bowl and put the yoghurt mass over them.

### EXAMPLE 18

### Confectionery

Recipe for milk chocolate

| | | |
|---|---|---|
| 40 | g | of sugar |
| 12 | g | of cocoa mass |
| 20 | g | of cocoa butter |
| 5 | g | of butyrate ester or microencapsulated butyrate ester |
| 20 | g | of whole milk powder |
| 2 | g | of hazelnut paste |
| 0.9 | g | of lecithin |
| 0.1 | g | of vanillin |

Make the sugar into a homogeneous mixture with the cocoa mass, half of the cocoa butter, whole milk powder and the hazelnut paste in a chocolate mixer. Mill the mixture to the desired fineness. Conch the remaining cocoa butter and rolled cocoa powder at a maximum of 70° C. for approximately 18-24 hours in the conch. Add the lecithin, butyrate ester and vanillin one hour before the end of the conching. Tempering of the mass and tableting.

### EXAMPLE 19

### Baked Products

Recipe for croissants

| | | |
|---|---|---|
| 25 | g | of yeast |
| 250 | g | of cream |
| 50 | g | of butyrate ester or microencapsulated butyrate ester |
| 50 | g | of sugar |
| 400 | g | of wheat flour type 550 |
| 0.15 | g | of salt |
| 200 | g | of margarine |
| 50 | g | of egg yolk |

Stir the yeast, lukewarm cream, a pinch of salt and 1 pinch of flour. Allow the dough to rise for 10 min. Knead with further additives and allow to rise for 20 min. Knead the dough thoroughly, roll out, cut out 15 triangles and roll up to give croissants. Briefly allow to rise and bake at 200° C. for 10 min.

### LITERATURE

Bach Knudsen, K. E., Serena, A., Canibe, N., Juntunen, K. S.: New insight into butyrate metabolism. Proc. Nutr. Soc. (2003) 62:81-86.
Cummings, J. H., Beatty, E. R., Kingman, S. M., Bingham, S. A., Englyst, H. N. Digestion and physiological properties of resistant starch in the human large bowel. Br J Nutr (1996) 75:733-47.
Cummings, J. H., Rombeau, J. L., Sakata, T. (Ed.): Physiological and clinical aspects of short chain fatty acids. Cambridge University Press, GB, 1995.
Cummings, J H, MacFarlane, G T, Englyst, H N: Prebiotic digestion and fermentation. Am J Clin Nutr (2001) 73:415 S-20S.
Desmet, G, Brazier, M, Cerutti, J, Chany, C, Arnould-Guerin, M L: Pharmacokinetics of butyric acid derivative with xylitol. Eur J Drug Metab Pharmacokinet (1991) Spec No. 3:348-51.
Newmarket, H L, Lupton, J R, Young, C W: Butyrate as a differentiating agent: pharmacokinetics, analogues and current status. Cancer Letters (1994) 78:1-5.
Pouillart, P., Cerutti, I, Ronco, G, Villa, P, Chany, C: Enhancement by stable butyrate derivatives of antitumor and antiviral actions of interferon. Int. J. Cancer (1992) 51(4): 596-601.
Rombeau, J L, Reilly, K J, Rolandelli, R H in: Cummings, J H, Rombeau, J L, Sakata, T (Ed.). Physiological and clinical aspects of short chain fatty acids. Cambridge University Press, GB (1995): 401-425.
Santini, V., Scappini, B., Gozzini, A., Grossi, A., Villa, P., Ronco, G., Douillet, O., Pouillart, P., Bernabei, P. A., Rossi Ferrini, P.: Butyrate-stable monosaccharide derivatives induce maturation and apoptosis in human acute myeloid leukemia cells. Br J Haematol (1998) 101(3): 529-38.
Scheppach W, Sommer H, Kirschner T, Paganelli G M, Bartram P, Christl S, Richter, F, Dusel G, Kasper H.: Effect of butyrate enemas on the colonic mucosa in distal ulcerative colitis. Gastroenterology (1992) July 103(1): 51-6.
Schmidt, M G, Soergel, K H, Wood, C M: Absorption of short chain fatty acids from the human jejunum. Gastroenterology (1976) 70:211-5.
Vernia, P, Montelone, G, Grandinetti, G, Villotti, G, Di Giulio, E, Frieri, G, Marcheggiano, A, Pallone, F, Caprilli, R, Torsoli, A: Combined oral sodium butyrate and mesalazine treatment compared to oral mesalazine alone in ulcerative colitis; randomized, double-blind, placebo-controlled pilot study. Dig Dis Sci (2000) May 45(5): 976-981
Wächtershäuser, A., Stein, J: Rationale for the luminal provision of butyrate in intestinal diseases. Eur J Nutr (2000) 39:164-171.
Wolin M J. Fermentation in the rumen and human large intestine. Science. (1981) 25; 213(4515): 1463-8.

1-20. (canceled)

21. A method for at least one of treating and preventing diseases of the gastrointestinal tract in the body of human and animal subjects, wherein said method comprises supplying a subject in need thereof with a butyric acid ester or a mixture of such esters in an amount sufficient to provide a therapeutic dosage of butyrate to the gastrointestinal tract of the subject.

22. The method according to claim 21, wherein the large intestine of the subject is provided with said butyrate.

23. The method according to claim 21, wherein the posterior large intestine of the subject is provided with said butyrate.

24. The method according to claim 21, wherein at least one said butyric acid ester has a degree of substitution (DS) of 3 to 4.

25. The method according to claim 21, wherein at least one said butyric acid ester is partially esterified.

26. The method according to claim 24, wherein the partially esterified butyric acid ester had a degree of esterification of 50% to 80%.

27. The method according to claim 21, wherein the butyric acid ester is selected from the group consisting of tributyrylsorbitol and tetrabutyrylsorbitol.

28. The method according to claim 21, wherein the butyric acid ester or the mixture thereof is administered orally or enterally to the gastrointestinal tract of the subject.

29. The method according to claim 27, wherein the butyric acid ester or the mixture is administered in microencapsulated form.

30. The method according to claim 28, wherein the microencapsulated form is prepared by a method selected from the group consisting of spray drying, freeze drying, fluidized bed drying, fluidized bed agglomeration, extrusion and atomization.

31. The method according to claim 27, wherein the butyric acid ester or the mixture is administered in macroencapsulated form.

32. A method of producing a medicament for at least one of treating and preventing diseases of the gastrointestinal tract in the body of human and animal subjects, wherein the medicament is formed with the butyric acid ester or mixture thereof according to claim 21 in an amount sufficient to provide a therapeutic dosage of butyrate to the gastrointestinal tract of a subject to which said medicament is administered.

33. The method according to claim 31, wherein the butyric acid ester or mixture thereof is in a microencapsulated or macroencapsulated form.

34. A pharmaceutical composition comprising a butyric acid ester of a mixture thereof in an amount sufficient to provide a therapeutic dosage of butyrate to the gastrointestinal tract of a subject when the pharmaceutical composition is administered to the subject.

35. A method for the production of a foodstuff, said method comprises including within said foodstuff the butyric acid ester or mixture thereof according to claim 21 in an amount sufficient to provide a therapeutic dosage of butyrate to the gastrointestinal tract of a subject consuming said foodstuff.

36. A composition comprising:
(a) at least one butyric acid ester or a combination thereof according to claim 21; and
(b) at least one further constituent selected from the group consisting of
i. carbohydrate polyols;
ii. carbohydrates;
iii. soluble and/or insoluble bulk materials;
iv. short-chain fatty acids;
v. butyric acid glycerol esters; and
vi. acylated starch.

37. The composition according to claim 35, wherein the further constituent is selected from:
i. mannitol, sorbitol, xylitol, lactitol, maltitol, erythritol, isomalt, 1,6-GPS, 1,1-GPS, 1,1-GPM, hydrogenated starch hydrolyzates, hydrogenated glucose syrups and mixtures thereof;
ii. monosaccharides, disaccharides, oligosaccharides, polysaccharides and mixtures thereof;
iii. soluble and/or insoluble bulk materials, in particular prebiotic and/or butyrogenic bulk materials, resistant starch, modified starch, polydextrose, fructooligosaccharides, galactooligosaccharides, trans-galactosylated oligosaccharides such as 6′-galactosyllactose or 4′-galactosyllactose, lactulose, lactobionic acid, maltobionic acid, xylooligosaccharides, lactosucrose, maltooligosaccharides, isomalto-oligosaccharides, gentiooligosaccharides, glucosylsucrose, soybean oligosaccharides, chitooligosaccharides, chitosan oligosaccharides, pectin, pectin oligosaccharides, condensed oligosaccharides, caramel products, galactomannan oligosaccharides, fucose-containing oligosaccharides, fucose derivative-containing oligosaccharides, pyrodextrin, partially hydrolyzed guar gum, a variant thereof obtained by partial hydrolysis, hydrogenation, oxidation, enzymatic or chemical modification of saccharides, and fibrous substances, in particular from oats, wheat, vegetables such as the tomato or pea, fruit such as apples and fruit berries, sugar beet, fruit of the carob tree or cellulose;
iv. butyric acid, propionic acid, acetic acid, lactic acid;
v. glycerol tributyrate, glycerol dibutyrate, glycerol monobutyrate; and
vi. butyrylated starch.

38. The composition according to claim 35, wherein the composition is a foodstuff and is selected from the group consisting of:
(i) dairy produce and dairy products including cheese, butter, yoghurt, kefir, quark, sour milk, buttermilk, cream, condensed milk, dried milk, whey, milk sugar, milk protein, mixed milk, half-fat milk, mixed whey or butterfat products or preparations;
(ii) blancmange, cream, mousse and other desserts;
(iii) butterfat products, mixed fat products, edible fats and edible oils;
(iv) baked products including bread including cookies and fine baked products, long-life baked products, cookie products and waffles;
(v) spreads, including fat-containing spreads, margarine products and shortening;
(vi) instant products and broth products;
(vii) fruit products or preparations including jams, marmalades, jellies, fruit preserves, fruit pulp, fruit pith, fruit juices, fruit juice concentrates, fruit nectar and fruit powder;
(viii) cereals, muesli and cereal mixtures, and ready prepared cereal-containing products including muesli bars and breakfast products;
(ix) nonalcoholic drinks, drink basic materials and drink powder;
(x) confectionery including chocolates, hard caramels, soft caramels, chewing gum, candy, fondant products, jelly products, licorice, marshmallow products, flakes, compressed sweets, candied fruit, brittle, nougat products, ice cups, marzipan, ice cream; and
(xi) a dietetic special food and enteral food derived therefrom.

39. The composition according to claim 35, wherein the composition is a feed and wherein the feed is selected from the group consisting of animal food, a premix for animal food, starch-rice feed, concentrated feed and mash.
